(19) United States
(12) Patent Application Publication
(10) Pub. No.: US 2020/0365254 A1
(43) Pub. Date: Nov. 19, 2020

(54) Medication Management Among A Network of Medical Entities

(71) Applicant: CareFusion 303, Inc., San Diego, CA (US)

(72) Inventors: Neal Zech, San Diego, CA (US); Jitendra Urankar, San Diego, CA (US); Karthik Ranganathan, San Diego, CA (US); Monica Wyly, San Diego, CA (US); Beth Lorden, San Diego, CA (US); Marcy Draves, San Diego, CA (US); Marla Madsen, San Diego, CA (US)

(21) Appl. No.: 15/931,517

(22) Filed: May 13, 2020

Related U.S. Application Data
(60) Provisional application No. 62/847,744, filed on May 14, 2019.

(51) Int. Cl. G16H 40/20; G16H 20/10; G16H 10/60; G16H 40/63; G16H 40/67

(52) U.S. Cl. CPC G16H 40/20; G16H 20/10; G16H 40/67; G16H 40/63; G16H 10/60

(57) ABSTRACT

Features relating to a medication management module for use in a network of medical entities are provided. Aspects of the current subject matter provide for tracking or otherwise managing medication inventory at the medical entities of the network. Additional aspects relate to sharing medication inventory information among entities of the network to, for example, enable borrowing of medication among entities. Other aspects relate to predicting availability of medication at the entities of the network. The medication management module provides visibility of medication inventory throughout the network of the medical entities to facilitate and/or cause sharing of medication.

### CROSS-REFERENCE TO RELATED APPLICATIONS

This application claims priority to and the benefit of U.S. Provisional Patent Application No. 62/847,744 which is entitled “MEDICATION MANAGEMENT AMONG A NETWORK OF MEDICAL ENTITIES,” and filed on May 14, 2019, the disclosure of which is incorporated herein by reference for all purposes.

### TECHNICAL FIELD

The current subject matter described herein relates generally to medication management and more particularly to managing medication inventory among a network of medical entities.

### BACKGROUND

Maintaining an accurate inventory of medication is of importance at a medical entity to enable the medical entity to provide proper and timely patient care. Among a network of medical entities, it may be desirable to have a consistent approach for medication inventory.

### SUMMARY

Aspects of the current subject matter relate to medication management in a network of medical entities. Aspects of the current subject matter provide for tracking or otherwise managing medication inventory at the medical entities of the network.

According to aspects of the current subject matter, a computer-implemented method includes receiving, at a medication management module and from a first processing device associated with a first medical entity, a medication identifier associated with a medication; creating, by the medication management module and based at least partially on the medication identifier, a medication record, wherein the medication record includes data linked to the medication identifier and a first medical entity identifier associated with the first medical entity, wherein the medication record further includes data linked to a second medical entity identifier associated with a second medical entity, and wherein the first medical entity and the second medical entity are in different locations; receiving, by the medication management module, use data related to the medication; updating, by the medication management module, the medication record in response to the received use data; and causing, based on the medication record, a sharing of the medication between the first medical entity and the second medical entity.

In an inter-related aspect, a system includes at least one data processor; and at least one memory storing instructions which, when executed by the at least one data processor, result in operations including receiving, from a first processing device associated with a first medical entity, a medication identifier associated with a medication; creating, based at least partially on the medication identifier, a medication record, wherein the medication record includes data linked to the medication identifier and a first medical entity identifier associated with the first medical entity, wherein the medication record further includes data linked to a second medical entity identifier associated with a second medical entity, and wherein the first medical entity and the second medical entity are in different locations; receiving use data related to the medication; updating the medication record in response to the received use data; and causing, based on the medication record, a sharing of the medication between the first medical entity and the second medical entity.

In an inter-related aspect, a non-transitory computer-readable storage medium including program code, which when executed by at least one data processor, causes operations including receiving, at a medication management module and from a first processing device associated with a first medical entity, a medication identifier associated with a medication; creating, by the medication management module and based at least partially on the medication identifier, a medication record, wherein the medication record includes data linked to the medication identifier and a first medical entity identifier associated with the first medical entity, wherein the medication record further includes data linked to a second medical entity identifier associated with a second medical entity, and wherein the first medical entity and the second medical entity are in different locations; receiving, by the medication management module, use data related to the medication; updating, by the medication management module, the medication record in response to the received use data; and causing, based on the medication record, a sharing of the medication between the first medical entity and the second medical entity.

In some variations, one or more of the features disclosed herein including the following features can optionally be included in any feasible combination. The medication identifier may be manually entered and/or received from a scanning or optical character recognition (OCR) device in communication with the medication management module. The first medical entity identifier and/or second medical entity identifier may be recognized by the medication management module and/or received by the medication management module. The data linked to the medication identifier may be accessible by the medication management module. The data linked to the medication identifier may include medication name, strength, form, volume, medication quantity, expiration date, and/or lot number. The computer-implemented method may further include determining, by the medication management module, that a current date is later than the expiration date; generating, by the medication management module and in response to the determining, an alert; and sending, by the medication management module, the alert to the first processing device, the second processing device, and/or a user. The use data may include quantity used, quantity wasted, date, patient, medical provider, and/or witness. The use data may be provided to the medication management module from a processing device associated with the medical entity. The computer-implemented method may further include causing a sharing of the medication by providing, by the medication management module, at least a portion of the medication record to the second processing device; receiving, by the medication management module and from the second processing device, a selection of a quantity of the medication to be borrowed from the first medical entity; and updating, by the medication management module, the medication record to reflect the borrowed quantity of the medication. The at least a portion of the medication record may be provided to the second medical entity via a display window of a graphical user interface of the second processing device. The selection of the quantity of the medication to be borrowed may be provided via use of a selection tool of the graphical user interface. The medication record may be updated in response to receipt, at the medication management module, of a confirmation signal from the first processing device and/or the second processing device. The computer-implemented method may further include receiving, by the medication management module and from the second processing device, an indication of a returned quantity of the medication borrowed from the first medical entity; and updating, by the medication management module, the medication record to reflect the returned quantity of the medication. The computer-implemented method may further include identifying, by the medication management module, a number of doses of the medication required for a predefined time period at the medical entity; generating, by the medication management module and in response to a determination that the number of doses is not available, a warning; and sending, by the medication management module, the warning to the first processing device and/or the second processing device. The warning may be provided on a display window of a graphical user interface of the first processing device and/or the second processing device. The required number of doses may be identified from scheduling information accessible to the medication management module. The warning may indicate an additional number of doses to satisfy the required number of doses. The computer-implemented method may further include sending, by the medication management module and to a medication ordering module, a request for the additional number of doses. The computer-implemented method may further include providing, by the medication management module and to the first processing device and/or the second processing device, a recommended adjustment to the medication.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a system diagram illustrating a computing landscape consistent with implementations of the current subject matter;

FIGS. 2A and 2B are exemplary display windows of a user interface for tracking medication inventory consistent with implementations of the current subject matter;

FIGS. 3A-3E are exemplary display windows of a user interface for sharing medication inventory consistent with implementations of the current subject matter;

FIGS. 4A-4D are exemplary display windows of a user interface for predicting availability of medication consistent with implementations of the current subject matter;

FIG. 5 is a flowchart illustrating a process of managing medication inventory among a network of entities consistent with implementations of the current subject matter; and

FIG. 6 depicts a block diagram illustrating a computing system consistent with implementations of the current subject matter.

When practical, similar reference numbers denote similar structures, features, or elements. Exemplary data for clinics, clinicians, or patients are realistic in form but not content and are not intended to represent actual clinics, clinicians, or patients. These values are included to illustrate how actual values may be represented in certain implementations.

### DETAILED DESCRIPTION

Aspects of the current subject matter relate to medication management in a network of medical entities, for example, clinics, doctor offices, medical centers, homecare facilities, hospitals, long term care facilities, other medical facilities, and/or the like. Aspects of the current subject matter provide for tracking or otherwise managing medication inventory at the medical entities of the network. Additional aspects relate to sharing medication inventory information among the medical entities of the network to, for example, enable borrowing of medication among entities. Other aspects relate to predicting availability of medication at the medication entities of the network.

Aspects of the current subject matter may, in some instances, be utilized in non-acute (i.e., non-hospital) settings. As non-acute settings may encompass a wide range of various entities, ranging from sophisticated medical centers to more basic clinics, with a wide range of resources, it may be difficult for the various entities to not only track their own inventory of medication but to also share the medication inventory among entities in the network. Automated medication and/or dispensing cabinets that may be available in acute (i.e., hospital) settings may not be readily available for various non-acute medical entities and may also be overly complex, resource intensive, and unnecessary for non-acute implementations. Aspects of the current subject matter address these and other limitations by providing a system and method for medication management among the network of medical entities. Although aspects may be described with respect to non-acute settings, implementations of the current subject matter are not limited to non-acute settings and may be applicable in acute settings.

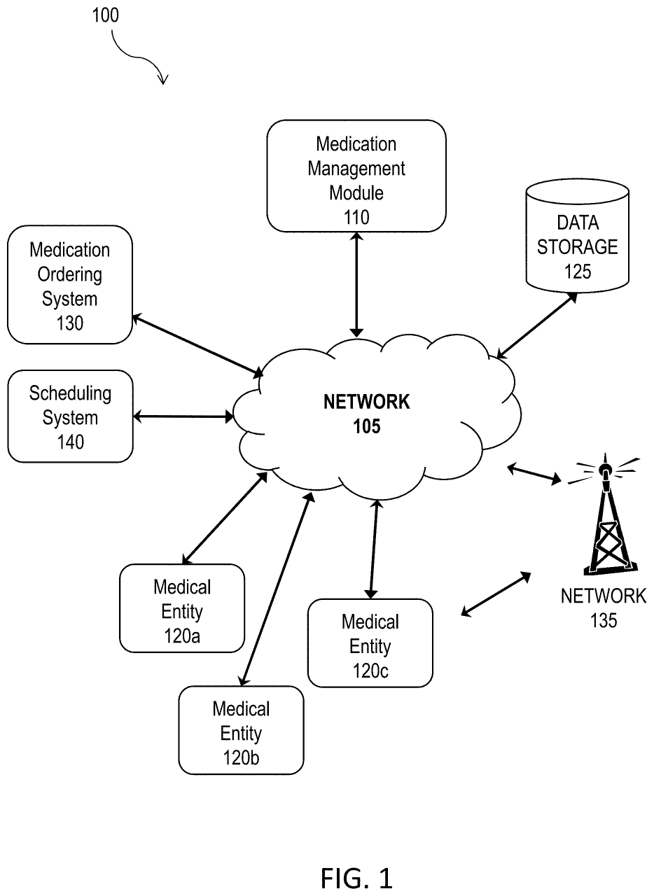

FIG. 1 is a system diagram illustrating a computing landscape 100 within a healthcare environment in which various aspects of the current subject matter may be employed. Various devices and systems, both local to the healthcare environment and remote from the healthcare environment, may interact via at least one computing network 105. This computing network 105 may provide any form or medium of digital communication connectivity (i.e., wired or wireless) amongst the various devices and systems. Examples of communication networks include a local area network (“LAN”), a wide area network (“WAN”), and the Internet. In some cases, one or more of the various devices and systems may interact directly via peer-to-peer coupling (either via a hardwired connection or via a wireless protocol such as Bluetooth or WiFi). In addition, in some variations, one or more of the devices and systems communicate via a public land mobile network (PLMN). The computing network 105 may include on premise, hosted, and/or cloud-based infrastructure.

In particular, aspects of the computing landscape 100 may be implemented in a computing system that includes a medication management module 110 (e.g., a middleware component or application server) and a plurality of medical entities 120a,b,c. Each medical entity 120a,b,c may be associated with a particular location and a non-acute or acute setting. The computing landscape 100 may also include a medication ordering system 130 and a scheduling system 140. The medication management module 110, the medical entities 120a,b,c, the medication ordering system 130, and the scheduling system 140 are generally remote from each other and may interact through the communications network 105. The relationship of the medical entities 120a,b,c and the medication management module 110 arises by virtue of computer programs running on the respective processing devices and having a client-server relationship to each other. The medical entities 120a,b,c may include any of a variety of computing platforms that include local applications for providing various functionality within the healthcare environment. The medical entities 120a,b,c may include one or more processing devices, for example, desktop computers, laptop computers, tablets, mobile devices, other computers with touch-screen interfaces, scanning devices, or the like. The one or more processing devices of the medical entities 120a,b,c may have a graphical user interface or a Web browser through which a user may interact with various implementations of the subject matter described herein. The local applications may be self-contained in that they do not require constant network connectivity.

A variety of applications may be executed on the various devices and systems within the computing landscape 100, for example, a medical ordering application, a scheduling application, electronic health record applications, data set editor applications, billing applications, and the like.

The network 105 may be coupled to one or more data storage systems 125. The data storage systems 125 may include databases providing physical data storage within the healthcare environment or within a dedicated facility. In addition, or in the alternative, the data storage systems 125 may include cloud-based systems providing remote storage of data in, for example, a multi-tenant computing environment and/or the like. The data storage systems 125 may also include non-transitory computer readable media.

The medical entities 120a,b,c may communicate directly via the network 105 and/or they may communicate with the network 105 via an intermediate network 135, for example, a cellular data network or a public land mobile network (PLMN).

Aspects of the current subject matter relate to providing visibility of medication inventory throughout the network of the medical entities 120a,b,c to facilitate and/or cause sharing of medication within the network. The medication management module 110 may cause sharing of a medication between medical entities 120a,b,c, by providing the ability to check inventory levels of the medication at other locations, transfer of a quantity of the medication from another location, and update the inventory level for both locations when the medication is transferred. Consistent with implementations of the current subject matter, the medication management module 110 may run a medication management application that tracks inventory levels for a medication within the network. The medication management application may link the medication with associated information, for example, identification of the medication, identification of one or more entities at which the medication is located, and quantity (i.e., inventory level) of the medication at the one or more entities. The associated information is stored and can be presented via a user interface of a computing device in the network for user review.

Consistent with implementations of the current subject matter directed to tracking medication inventory, the medication management module 110 may receive from one of the medical entities 120a,b,c a medication identifier associated with a medication. For example, the medical entity 120a,b,c may scan with a scanning device or a mobile device a code (e.g., barcode, quick read code, radio frequency identifier, etc.) associated with the medication, or utilize optical character recognition (OCR) technology to read information from a medication label, or a user may manually enter on a processing device a number, for example, a serial number or other identifier, associated with the medication. In some instances, the medication may be included in a lot. The lot may include a plurality of doses of the medication having a common characteristic such as manufacturing event time.

The medication management module 110 may, upon receipt of the medication identifier, create or update a medication record associated with the medication. The medication record may include data linked to the medication identifier. For example, when the medication is packaged, various pieces of information related to the medication may be associated with the medication identifier. The association may be done by a manufacturer, distributer, and/or the like. The data linked to the medication identifier may be accessible by the medication management module 110 via, for example, the data storage system 125. The data linked to the medication identifier may include medication name, lot number, distributor source, medication type, dosage or strength, medication quantity, and/or expiration date. For example, in instances in which the medication includes a plurality of doses, the linked data may indicate the number of doses. The medication record may also include a medical entity identifier associated with the medical entity 120a,b,c that provided the medication identifier. This allows for the medication management module 110 to track medication inventory per medical entity 120a,b,c. The medical entity identifier may be recognized by the medication management module 110. For example, the medication management module 110 may automatically receive the medical entity identifier upon receiving the medication identifier from the medical entity 120a,b,c. Alternatively or additionally, the medical entity 120a,b,c may provide the medical entity identifier to the medication management module prior to sending the medication identifier or with the medication identifier.

Once the medication record is created and stored (e.g., in the data storage system 125) by the medication management module 110, the medication management module 110 may subsequently receive use data related to the medication. For example, when the medical entity 120a,b,c administers a dose of the medication to a patient, the medical entity 120a,b,c may provide (through one of the processing devices) use data indicating relevant information related to the administration of the dose. A scanning device or a mobile device may scan the code (e.g., barcode, quick read code, radio frequency identifier, etc.) associated with the medication, or utilize OCR technology to read information from the medication label, or a user may manually enter on a processing device a number associated with the medication. The user may also provide additional information, such as the patient to whom the dose is to be administered and the medical provider who accessed the medication from the system. In some implementations, the use data may include, for example, quantity used, date, time, patient, medical provider, and/or the like. Some of the use data may be automatically provided to or determined by the medication management module 110. For example, if a particular user is logged into the application, the medication management module 110 may associate the user as the medical provider without further input from the user.

The medication management module 110 uses the received use data to update the medication record. As patients are now tied to the medication via the medication record, patients or clinicians may be easily identified and contacted in case of recalls or other issues with respect to the medication.

The medication record may also be used to process and send various alerts. For example, with respect to expiration of the medication, the medication management module 110 may determine that an expiration date is within a predefined period of time and may accordingly send an alert message (e.g., an email message, a text message, a message in a pop-up window, or the like) to one or more processing devices of the medical entity 120a,b,c. The predefined period of time may be a standard value (two weeks, one week, five days, three days, one day, etc.) and/or may be established by the medical entity 120a,b,c. The medication management module 110 may determine that a current date is past the expiration date of the medication, and may accordingly generate and send an alert to warn the medical entity 120a,b,c. A particular processing device may be selected to receive the alert. The selection may be based on location of the device in relation to a location of the medication referenced in the alert. The selection may be based on one or more clinicians associated with the device. For example, the medication may be a specialized drug used for certain care areas. In such instances, the alert may be transmitted to the processing device(s) of those clinicians associated with the specified care areas. In some implementations, it may further identify those clinicians who are currently working based on, for example, a time and attendance schedule for the medical entity 120a,b,c.

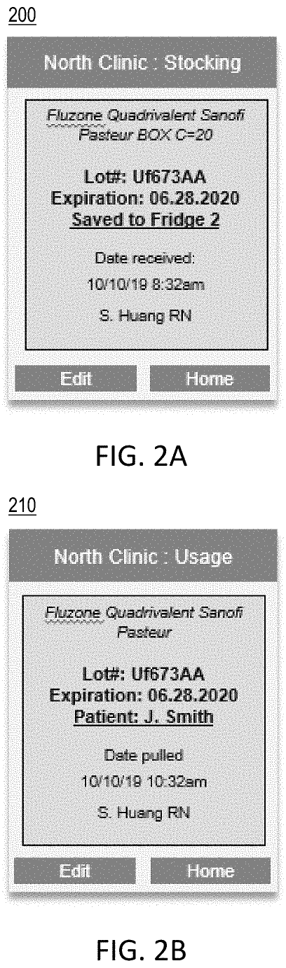

FIGS. 2A and 2B are exemplary display windows 200 and 210 respectively of a user interface for tracking medication inventory consistent with implementations of the current subject matter. For example, the display windows 200 and 210 may be provided by the medication management application. The display window 200 in FIG. 2A is an example of a display that a user at the medical entity 120a,b,c may have access to when scanning or entering a medication identifier. A name of the medical entity 120a,b,c, may be included in the display window 200, as well as the name of the medication, the lot number, the expiration date, a location in which the medication is being stored, the date and time at which the medication is being entered or processed, and the medical provider. As described herein, the medication management module 110 may use this data to create the medication record.

The display window 210 in FIG. 2B is an example of a display that may be presented to a user at the medical entity 120a,b,c when providing use data related to the medication, for example, when administering a dose of the medication. In addition to the name of the medical entity 120a,b,c, the name of the medication, the lot number, the expiration date, and the medical provider, the name of the patient and a date and time at which the medication is accessed may be included in the display window 210. As described herein, the medication management module 110 may use the use data to update the medication record associated with the medication. Attributes of the display window 210 may be adjusted to provide additional information such as expiration alerts to the user. For example, if a medication is expired, the display window 210 may present an icon or change a background color to draw attention to the expiration condition.

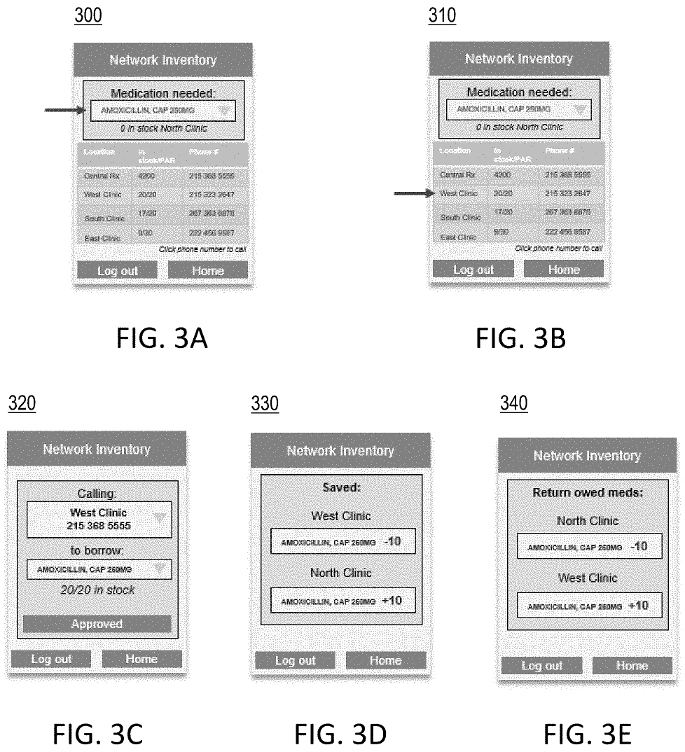

Consistent with additional aspects of the current subject matter, the medication management application run by the medication management module 110 may facilitate providing visibility of medication inventory throughout the network of the medical entities 120a,b,c to facilitate and/or cause sharing of medication within the network. For example, the medication management application may provide a display window on a user interface indicating to each of the medical entities 120a,b,c a quantity of a particular medication at each of the medical entities 120a,b,c. An example of features and a user interface are discussed with reference to FIG. 3A.

The medication management application may further facilitate contact between the medical entities 120a,b,c, and may additionally provide for medication borrowed amounts and medication returned amounts to be indicated, such as shown and discussed with reference to FIGS. 3B, 3C, 3D, and 3E. The medication management module 110 may use this information to update the medication records, as further described herein. The medication management module 110 may facilitate providing visibility of medication inventory throughout the network of medical entities 120a,b,c to facilitate managing network level inventory levels and configuring par levels for medical entities within the network. For example, if the medication management module 110 detects an inventory level for a medication that is consistently above or below a threshold, the medication management module 110 may suggest or cause an adjustment to the par level of the medication.

The medication management module 110 may provide the medication record or portions thereof including the medication name, the quantity, and the associated medical entity 120a,b,c to each of the medical entities 120a,b,c. The medication management module 110 may receive from a processing device of one of the medical entities 120a,b,c a selection of a particular medical entity 120a,b,c, a selection or indication of the medication to be borrowed, and a selection or indication of a quantity of the medication to be borrowed. The medication management module 110 may update the medication record associated with the medication to be borrowed to reflect the borrowed quantity of the medication. That is, the medication management module 110 may accordingly, in the medication record, decrease the quantity of the medication at the medical entity 120a,b,c from which the medication is borrowed. The borrow request may be transmitted from the requesting entity to the entity having the requested amount. In some implementations of the current subject matter, the medication management module 110 may update the medication record in response to a confirmation signal or acknowledgment from the medical entity 120a,b,c from which the medication is being borrowed.

The medication management module 110 may provide on the user interface one or more selection tools to select or otherwise indicate the medication of interest, the medical entity 120a,b,c of interest, and/or the quantity to be borrowed from the selected or indicated medical entity 120a,b,c.

The medication management module 110 may subsequently receive, from the medical entity 120a,b,c that borrowed the medication, an indication of a returned quantity of the medication borrowed. For example, the medical entity 120a,b,c may have borrowed 20 units of the medication but only needed 10 units. The medical entity 120a,b,c may wish to return the unused units to the medical entity 120a,b,c from which the medication was borrowed. To accordingly update the medication record, one or both of the medical entities 120a,b,c may send or provide an indication to the medication management module 110 so that an accurate medication inventory for each entity within the network is maintained. Upon receipt of the indication, the medication management module may accordingly update the medication record to reflect the returned quantity of the medication.

FIGS. 3A, 3B, 3C, 3D, and 3E are exemplary display windows 300, 310, 320, 330, and 340, respectively, of a user interface for sharing medication inventory consistent with implementations of the current subject matter. In particular, the exemplary display windows 300-340 illustrate an example series of display windows that may be generated by the medication management application to facilitate and/or cause sharing of medication between the medical entities 120a,b,c.

The display window 300 of FIG. 3A and the display window 310 of FIG. 3B indicate a selection of a desired medication (through, for example, a dropdown bar) and associated information including the medical entities 120a,b,c that contain an inventory of the medication, the amount at each medical entity 120a,b,c, and a telephone number for each of the medical entities 120a,b,c. The telephone number may be provided to facilitate connecting the two medical entities for the medication sharing process. For example, by selection of a telephone number, the medication management module 110 may establish a telephone call between processing devices (e.g., mobile phones or other devices) of the medical entities. Other forms of communication may alternatively or additionally be used, such as messaging, text messaging, email, or the like. Moreover, a user is not required to select a telephone number or other contact information, but may instead initiate separate contact with the desired medical entity 120a,b,c.

The display window 320 of FIG. 3C indicates a summary of a selection made to initiate borrowing of a medication. A user may approve or confirm the selection to initiate transmission of the communication.

The medication management module 110 may provide the display window 330 of FIG. 3D to allow for the medical entity 120a,b,c borrowing and/or the medical entity 120a,b,c lending the medication to indicate the appropriate amounts to allow for the medication management module 110 to accordingly update the medication record. In some implementations, one medical entity 120a,b,c may need to indicate the borrowed information, while in other implementations both medical entities 120a,b,c may need to indicate the borrowed information.

The medication management module 110 may provide the display window 340 of FIG. 3E to allow for the medical entity 120a,b,c returning the medication and/or the medical entity 120a,b,c that lent the medication to indicate the appropriate amounts returned, thus allowing for the medication management module 110 to accordingly update the medication record.

Consistent with additional aspects of the current subject matter, the medication management application run by the medication management module 110 may facilitate predicting availability of medication at the medication entities 120a,b,c of the network. For example, the medication management module 110 may tie in scheduling information from, for example, the scheduling system 140 and medication inventory of the medical entities 120a,b,c to predict medication usage and highlight potential shortages. This allows for the medical entity 120a,b,c to attempt to procure the needed medication or reschedule one or more patients requiring the needed medication.

Consistent with implementations of the current subject matter, the medication management module 110 may identify a number of doses of the medication required for a predefined time period at the medical entity 120a,b,c. The required number of doses may be identified from scheduling information accessible to the medication management module 110 via, for example, the scheduling system 140. In some instances, the required number of doses may be obtained by the medication management module 110 from data or information provided from the medical entity 120a,b,c. The predefined time period may be a set or established time period, or may be a desired time period established by the medical entity 120a,b,c. For example, the medical entity 120a,b,c may want to verify bi-weekly, weekly, and/or daily if the medical entity 120a,b,c has a sufficient supply of the medication. In some implementations, the quantity may be referred to as the periodic automatic replenishment (PAR) level.

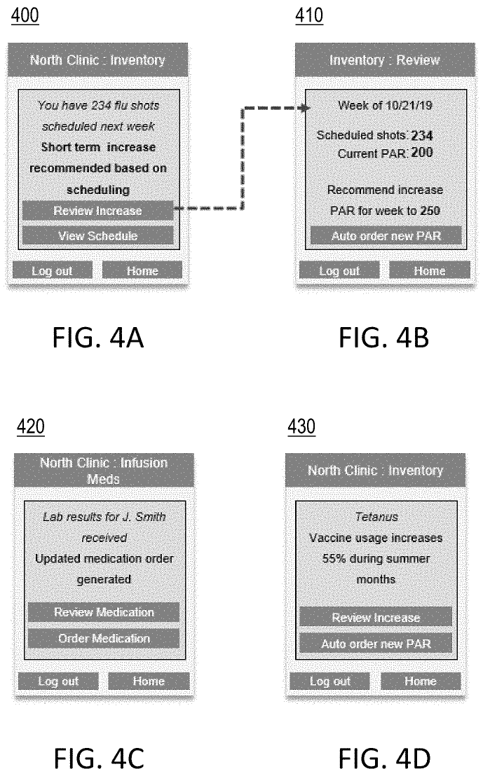

If the medication management module 110 determines that the number of doses is not available, the medication management module 110 may generate and send to the medical entity 120a,b,c a warning to indicate the insufficient number of doses (FIG. 4A). For example, the medication management module 110 may compare scheduling information with the medication inventory to determine if a sufficient number of doses are available. As one example, if the medical entity 120a,b,c has 150 flu vaccinations scheduled for a particular week, the medication management module 110 compares the 150 flu vaccination appointments with the number of remaining doses of the flu vaccine. The generated warning may be provided on a display window of a user interface of a processing device at the medical entity 120a,b,c. Alternatively and/or additionally, the generated warning may be sent from the medication management module 110 as an email message, text message, or voicemail message. Consistent with implementations of the current subject matter, the warning may indicate an additional number of doses needed to satisfy the required number of doses (FIG. 4B).

The medication management module 110 may send to a medication ordering module, for example, the medication ordering system 130, a request for the additional number of doses. The medication management module 110 may also add a buffer of at least one to account for unforeseen accidents or unexpected scheduling issues or errors.

Consistent with some implementations of the current subject matter, the medication management module 110 may provide a recommended adjustment to the medication inventory. For example, if 150 flu vaccinations are needed and the medical entity 120a,b,c has 125 flu vaccinations, the medication management module 110 may recommend an additional amount greater than or equal to 25 flu vaccinations. After receiving the additional 25 flu vaccinations, the medical entity 120a,b,c will have the 150 flu vaccinations needed. The recommended adjustment may be based on, for example, general availability of the medication, cost of the medication, historical trends of use of the medication, current medical circumstances, and/or predefined settings established for the network or by the medical entity 120a,b,c. As one example, if based on scheduling information it is determined that five doses of a particular medication are required and the medical entity 120a,b,c has three doses, the medication management module 110 may use a number of factors to recommend an adjustment to the medication inventory. For example, from historical information available from the data storage system 125, the medication management module 110 may determine that it is unlikely that more than a few extra doses will be required. As another example, if there is a known outbreak of a particular illness treatable by a particular medication, the medication management module 110 may determine that an increase in the medication inventory is necessary. In some instances, the medication management module 110 may provide a recommended adjustment of a predefined percentage, for example, 5%, 10%, 15%, 20%, 25%, etc. more than what is currently needed.

FIGS. 4A, 4B, 4C, and 4D are exemplary display windows 400, 410, 420, and 430, respectively, of a user interface for predicting availability of medication consistent with implementations of the current subject matter. In particular, the exemplary display windows 400-430 illustrate an example series of display windows that may be generated by the medication management application with respect to predicting medication usage and highlighting potential shortages for the medical entities 120a,b,c.

The display window 400 of FIG. 4A provides an example display used to highlight to a user at the medical entity 120a,b,c a predicted shortage of the medication based on available scheduling information. The display window 400 may include options to review a recommended adjustment and to view the schedule.

The display window 410 of FIG. 4B provides an example display in response to selection of the review a recommended adjustment. The display window 410 may include an option to order the recommended amount.

The display window 420 of FIG. 4C provides, consistent with an additional implementation of the current subject matter, information related to adjusting medication orders based on, for example, patient laboratory results or patient visit information. For example, the laboratory results or the patient visit may indicate a certain type of medication needed to treat the patient. The medication management module 110 may determine the type of medication directly from the laboratory results or from a physician's order. In some instances, the medication management module 110 may be provided with the needed medication. The display window 420 may include options to review the medication and order the medication.

The display window 430 of FIG. 4D provides, consistent with an additional implementation of the current subject matter, suggested adjustments based on various trends and/or circumstances, for example, a particular season. The display window 430 may indicate that a particular medication is in greater demand during a particular time or based on other known or predictable events, such as an outbreak of the flu or other illness. In some implementations, the system may analyze historic use information to identify adjustments to levels in anticipation of increased or decreased demand. For example, at the beginning of a school year, a pediatric clinic may see a spike in demand for a particular medication as parents get their children ready for the school year. This spike may be detected by comparing use for the medication over time. In some implementations, the prediction may be based on a comparison between similar acuity clinics or clinics offering a similar care type (e.g., pediatrics, geriatrics, general out-patient, etc.). The display window 430 may include options to review the suggested adjustment and transmit an order for the suggested adjustment. In some implementations, the adjustment may include transmitting a message to cancel an open order for a medication which is currently in high supply.

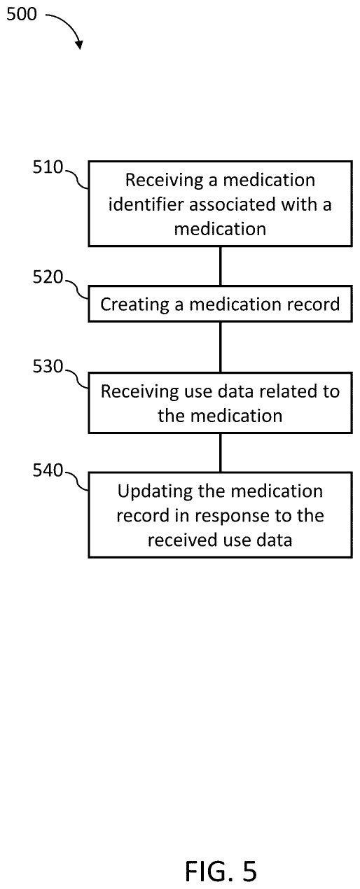

FIG. 5 depicts a flowchart illustrating a process 500 of managing medication inventory by the medication management module 110 among a network of entities 120a,b,c, consistent with implementations of the current subject matter.

At 510, the medication management module 110 may receive a medication identifier associated with a medication. The medication management module 110 may receive the medication identifier from a medical entity 120a,b,c. The medication management module 110 may also receive a medical entity identifier of the medical entity 120a,b,c that provides the medication identifier. The medication management module 110 may receive, from the medical entity 120a,b,c, the medical entity identifier prior to or along with the medication identifier. For example, the medical entity 120a,b,c may scan, with a scanning device or a mobile device, a code (e.g., a barcode, a quick read code, a radio frequency identifier, and/or the like) on and/or associated with the medication. A user may utilize an optical sensor (e.g., a camera) and optical character recognition technology to read the medication identifier from a medication label, a patient's chart, and/or a medical record. A user may manually enter the medication identifier on a processing device. The medication identifier may include letters, numbers, and/or punctuation. For example, a medication identifier may include a serial number and/or other identifiers associated with the medication.

At 520, the medication management module 110 may create, based at least partially on the medication identifier, a medication record. The medication record may be stored in the data storage system 125. The medication record may include data linked to the medication identifier and/or a medical entity identifier associated with the medical entity 120a,b,c. For example, when the medication is packaged, various attributes and/or pieces of information related to the medication may be associated with the medication identifier. The data linked to the medication identifier may include a medication name, a strength, a form, a volume, a lot number or lot code, a medication quantity, and/or an expiration date. The medication record may also include a medical entity identifier associated with the medical entity 120a,b,c that provided the medication identifier. The lot number or lot code may identify a lot. The lot may include a plurality of doses of the medication that have a common characteristic, such as a date and/or time of manufacture. The medication management module may use the medication record to track medication inventory available at the medical entity 120a,b,c. The medication management module 110 may use the medication identifier to obtain, from the medication record, the medical entity identifier of the medical entity 120a,b,c that provided the medication identifier. The medication record may include a manufacturer identifier, a distributor identifier, and/or the like, indicating a source for acquiring the medication. The medication management module 110 may access the medication record via the storage system 125. If the medication includes a plurality of doses, the medication record may indicate a number of doses.

At 530, the medication management module 110 may receive use data related to a usage of the medication. The use data may be received from the medical entity 120a,b,c in response to the medication being administered, such as in response to the medication being administered to a patient at the medical entity 120a,b,c. The use data may include a medication identifier. A user may scan the medication identifier using a scanning device or a mobile device (e.g., barcode, quick read code, radio frequency identifier, etc.) The user may utilize an optical sensor (e.g., a camera) and optical character recognition technology to read the medication identifier from a medication label, a patient's chart, and/or a medical record. The user may manually enter the medication identifier on a processing device. The use data may include a patient identifier linked to information about the patient to whom the dose is administered. The use data may include other information, such as a quantity used, a date and/or time the medication was administered, a medical provider, and/or the like. For example, when the medical entity 120a,b,c administers a dose of the medication to a patient, the medical entity 120a,b,c may provide (through one of the processing devices) use data indicating relevant information related to the administration of the dose. In some implementations, the receipt of the use data may be passive whereby one of the processing devices transmits the use data to the medication management module 110. In some implementations, the receipt of the use data may be scheduled whereby the medication management module 110 periodically queries one of the processing devices for the use data accordingly. In some implementations, the receipt of the use data may be triggered by an event detected by the medication management module 110, such as an update to an electronic medical record for a patient, detecting an entry into a waste log, and/or the like.

At 540, the medication management module 110 may update the medication record in response to the received use data. The medication management module may update the medication record on the data storage system 125. The medication management module 110 may update the medication record to update an inventory level for the medical entity 120a,b,c that used the medication. The medication management module 110 may obtain, from the use data, a medical entity identifier, a medication identifier, a quantity used, and a date and time when the medication was administered. The medication management module 110 may adjust the medication record associated with the medical entity 120a,b,c to reflect an updated medication quantity (e.g., inventory level.) For example, a value of the medication quantity may be decreased to account for the dose of the medication administered to the patient.

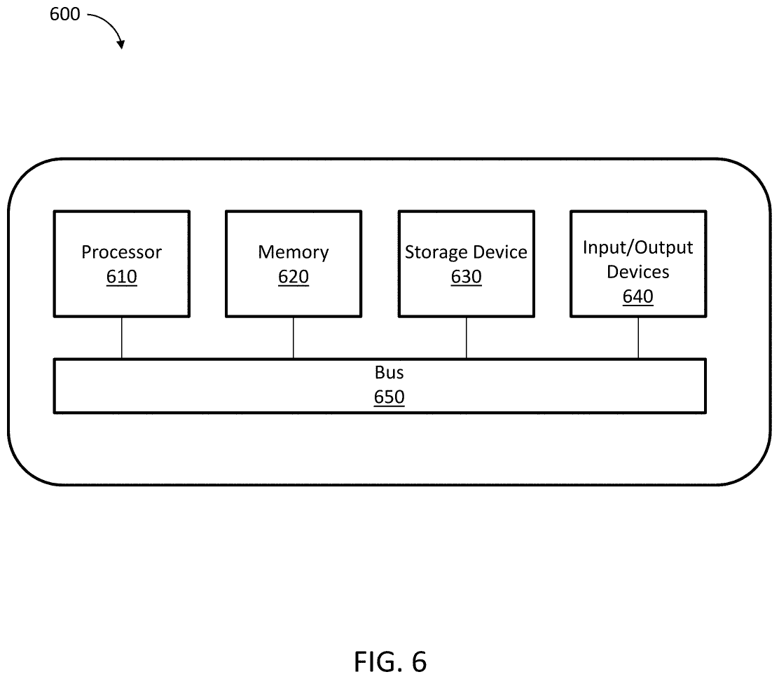

FIG. 6 depicts a block diagram illustrating a computing system 600 consistent with implementations of the current subject matter. Referring to FIG. 1, the computing system 600 can be used to implement the system 100 and/or any components therein.

As shown in FIG. 6, the computing system 600 can include a processor 610, a memory 620, a storage device 630, and input/output devices 640. The processor 610, the memory 620, the storage device 630, and the input/output devices 640 can be interconnected via a system bus 650. The processor 610 is capable of processing instructions for execution within the computing system 600. Such executed instructions can implement one or more components of, for example, the system 100. In some implementations of the current subject matter, the processor 610 can be a single-threaded processor. Alternately, the processor 610 can be a multi-threaded processor. The processor 610 is capable of processing instructions stored in the memory 620 and/or on the storage device 630 to display graphical information for a user interface provided via the input/output device 640.

The memory 620 is a computer readable medium such as volatile or non-volatile that stores information within the computing system 600. The memory 620 can store data structures representing configuration object databases, for example. The storage device 630 is capable of providing persistent storage for the computing system 600. The storage device 630 can be a floppy disk device, a hard disk device, an optical disk device, or a tape device, or other suitable persistent storage means. The input/output device 640 provides input/output operations for the computing system 600. In some implementations of the current subject matter, the input/output device 640 includes a keyboard and/or pointing device. In various implementations, the input/output device 640 includes a display unit for displaying graphical user interfaces.

According to some implementations of the current subject matter, the input/output device 640 can provide input/output operations for a network device. For example, the input/output device 640 can include Ethernet ports or other networking ports to communicate with one or more wired and/or wireless networks (e.g., a local area network (LAN), a wide area network (WAN), the Internet).

In some implementations of the current subject matter, the computing system 600 can be used to execute various interactive computer software applications that can be used for organization, analysis and/or storage of data in various (e.g., tabular) format (e.g., Microsoft Excel®, and/or any other type of software). Alternatively, the computing system 600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 640. The user interface can be generated and presented to a user by the computing system 600 (e.g., on a computer screen monitor, etc.).

One or more aspects or features of the subject matter described herein can be realized in digital electronic circuitry, integrated circuitry, specially designed ASICs, field programmable gate arrays (FPGAs), computer hardware, firmware, software, and/or combinations thereof These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

Although the disclosure, including the figures, described herein may describe and/or exemplify different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise” and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system, comprising:
at least one data processor; and
at least one memory storing instructions which, when executed by the at least one data processor, result in operations comprising:
receiving, at a medication management module and from a first processing device associated with a first medical entity, a medication identifier associated with a medication;
creating, by the medication management module and based at least partially on the medication identifier, a medication record, wherein the medication record comprises data linked to the medication identifier and a first medical entity identifier associated with the first medical entity, wherein the medication record further comprises data linked to a second medical entity identifier associated with a second medical entity, and wherein the first medical entity and the second medical entity are in different locations;
receiving, by the medication management module, use data related to the medication;
updating, by the medication management module, the medication record in response to the received use data; and
causing, based on the medication record, a sharing of the medication between the first medical entity and the second medical entity.

2. The system of claim 1, wherein the medication identifier is manually entered and/or received from a scanning or optical character recognition (OCR) device in communication with the medication management module.

3. The system of claim 1, wherein the first medical entity identifier is recognized by the medication management module and/or received by the medication management module, and wherein the second medical entity identifier is recognized by the medication management module and/or received by the medication management module.

4. The system of claim 1, wherein the data linked to the medication identifier is accessible by the medication management module.

5. The system of claim 1, wherein the data linked to the medication identifier comprises a medication name, a strength, a form, a volume, a medication quantity, an expiration date, and/or a lot number.

6. The system of claim 5, further comprising:
determining, by the medication management module, that a current date is later than the expiration date;
generating, by the medication management module and in response to the determining, an alert; and
sending, by the medication management module, the alert to the first processing device, the second processing device, and/or a user.

7. The system of claim 1, wherein the use data comprises a quantity used, a quantity wasted, a date, a patient, a medical provider, and/or a witness.

8. The system of claim 1, wherein the use data is provided to the medication management module from the first processing device and/or the second processing device.

9. The system of claim 1, wherein causing the sharing of the medication further comprises:
providing, by the medication management module, at least a portion of the medication record to the second processing device;
receiving, by the medication management module and from the second processing device, a selection of a quantity of the medication to be borrowed from the first medical entity; and
updating, by the medication management module, the medication record to reflect the borrowed quantity of the medication.

10. The system of claim 9, wherein the at least a portion of the medication record is provided to the second medical entity via a display window of a graphical user interface of the second processing device.

11. The system of claim 10, wherein the selection of the quantity of the medication to be borrowed is provided via use of a selection tool of the graphical user interface.

12. The system of claim 9, wherein the medication record is updated in response to receipt, at the medication management module, of a confirmation signal from the first processing device and/or the second processing device.

13. The system of claim 9, further comprising:
receiving, by the medication management module and from the second processing device, an indication of a returned quantity of the medication borrowed from the first medical entity; and
updating, by the medication management module, the medication record to reflect the returned quantity of the medication.

14. The system of claim 1, further comprising:
identifying, by the medication management module, a number of doses of the medication required for a predefined time period at the first medical entity and/or the second medical entity;
generating, by the medication management module and in response to a determination that the number of doses is not available, a warning; and
sending, by the medication management module, the warning to the first processing device and/or the second processing device.

15. The system of claim 14, wherein the warning is provided on a display window of a graphical user interface of the first processing device and/or the second processing device.

16. The system of claim 14, wherein the required number of doses is identified from scheduling information accessible to the medication management module.

17. The system of claim 14, wherein the warning indicates an additional number of doses to satisfy the required number of doses.

18. The system of claim 17, further comprising:
sending, by the medication management module and to a medication ordering module, a request for the additional number of doses; and
providing, by the medication management module and to the first processing device and/or the second processing device, a recommended adjustment to the medication.

19. A method, comprising:
receiving, from a first processing device associated with a first medical entity, a medication identifier associated with a medication;
creating, based at least partially on the medication identifier, a medication record, wherein the medication record comprises data linked to the medication identifier and a first medical entity identifier associated with the first medical entity, wherein the medication record further comprises data linked to a second medical entity identifier associated with a second medical entity, and wherein the first medical entity and the second medical entity are in different locations;
receiving use data related to the medication;
updating the medication record in response to the received use data; and
causing, based on the medication record, a sharing of the medication between the first medical entity and the second medical entity.

20. A non-transitory computer-readable storage medium including program code, which when executed by at least one data processor, causes operations comprising:
receiving, at a medication management module and from a first processing device associated with a first medical entity, a medication identifier associated with a medication;
creating, by the medication management module and based at least partially on the medication identifier, a medication record, wherein the medication record comprises data linked to the medication identifier and a first medical entity identifier associated with the first medical entity, wherein the medication record further comprises data linked to a second medical entity identifier associated with a second medical entity, and wherein the first medical entity and the second medical entity are in different locations;
receiving, by the medication management module, use data related to the medication;
updating, by the medication management module, the medication record in response to the received use data; and
causing, based on the medication record, a sharing of the medication between the first medical entity and the second medical entity.
